(19) United States
(12) Patent Application Publication
(10) Pub. No.: US 2010/0240745 A1
(43) Pub. Date: Sep. 23, 2010

(54) METHOD OF TREATMENT OF INFLAMMATION IN HYPERTENSIVE PATIENTS

(75) Inventors: Rosa Rosanna B. FLEMING, Ringoes, NJ (US); Brigitte Duquesroix, Cagnes sur mer (FR); William B. White, Canton, CT (US)

Correspondence Address: ARENT FOX LLP, 1050 CONNECTICUT AVENUE, N.W., SUITE 400, WASHINGTON, DC, 20036, US

(73) Assignee: NICOX S.A., Sophia Antipolis - Valbonne (FR)

(21) Appl. No.: 12/727,572

(22) Filed: Mar. 19, 2010

Related U.S. Application Data
(60) Provisional application No. 61/162,068, filed on Mar. 20, 2009.

(51) Int. Cl. A61K 31/216; A61P 29/00; A61P 19/02

(52) U.S. Cl. 514/509

(57) ABSTRACT

The present invention relates to the use of 4-(nitrooxy)-butyl-(S)-2-(6-methoxy-2-naphthyl)-propanoate (naproxcinod) for the treatment of pain and/or inflammation in patients with hypertension treated with renin-angiotensin system blocking agents.

The present invention relates to the use of 4-(nitrooxy)butyl(2S)-2-(6-methoxy-2-naphthyl)propanoate (naproxcinod) for the treatment of inflammation, in particular the treatment of signs or symptoms of musculo-skeletal disorders such as osteoarthritis, in hypertensive patients.

4-(nitrooxy)butyl(2S)-2-(6-methoxy-2-naphthyl)propanoate is a nitric oxide derivative releasing naproxen with reduced gastrointestinal and cardiovascular toxicity, and with analgesic, anti-inflammatory and antipyretic effects (Schnitzer T J. et al; Arthritis Rheum. 2005; 53:827-837).

WO 95/09831 discloses the synthesis of 4-(nitrooxy) butyl (2S)-2-(6-methoxy-2-naphthyl)propanoate and its anti-inflammatory, analgesic, anti-aggregating activities and its reduced gastrointestinal toxicity. WO 95/09831 does not mention the effect of 4-(nitrooxy)butyl(2S)-2-(6-methoxy-2-naphthyl) on blood pressure.

Osteoarthritis and hypertension are commonly observed co-morbidities in elderly patients and co-administration of non-steroidal anti-inflammatory drugs (NSAIDs) with antihypertensive agents is very common in clinical practice (Singh G. et al; Am J Manag Care. 2002; 8(15 suppl): S383-S391, Caporali R. et al. Semin Arthritis Rheum. 2005; 35 (Suppl 1):31-37, White W B. et al; Hypertension. 2004; 44:123-124).

NSAIDs are widely used to alleviate pain and inflammation but while they are considered relatively safe for acute and short-term use, there are well known adverse effects in chronic users. In particular, NSAIDs (eg, ibuprofen, indomethacin, and naproxen) could increase mean arterial pressure by as much as 5 to 6 mmHg in hypertensive patients. Pope J E. et al; Arch Intern Med. 1993; 153:477-484, Johnson A G. et al; Ann Intern Med. 1994; 121:289-300.

Moreover, it is well recognised that NSAIDs may also interfere with hypertension management in patients with osteoarthritis and pre-existing hypertension, especially those treated with agents that block the renin-angiotensin system, starting as early as 1 to 3 weeks of initiating NSAID therapy. In such cases, blood pressure destabilization, which may lead to hypertensive complications, commonly develops. Whelton A. et al, Am J Cardiol. 2002, 90:959-963; Morgan T O. et al, Am J Hypertens. 2000, 13:1161-1167; Solomon D H. et al; Hypertension. 2004; 44:140-145.

Based on the foregoing discussion, there is a recognised need for anti-inflammatory drugs to be used in the subpopulation of osteoarthritic patients with treated hypertension.

Surprisingly and unexpectedly, it was found that 4-(nitrooxy)butyl(2S)-2-(6-methoxy-2-naphthyl)propanoate does not induce blood pressure elevation in osteoarthritic hypertensive patients treated with at least a renin-angiotensin system blocking agent.

The present invention is therefore related to a method for the treatment of inflammation, in particular for the treatment of signs or symptoms of musculo-skeletal disorders such as osteoarthritis, comprising administering to a patient with hypertension, who is administered with at least one renin-angiotensin system blocking agent, a therapeutically effective amount of 4-(nitrooxy)butyl(25)-2-(6-methoxy-2-naphthyl)propanoate.

The present invention is further related to a method for the treatment of inflammation, in particular for the treatment of signs or symptoms of musculo-skeletal disorders such as osteoarthritis, comprising administering to a patient with hypertension, who is co-administered with at least a renin-angiotensin system blocking agent and with a diuretic, a therapeutically effective amount of 4-(nitrooxy)butyl(2S)-2-(6-methoxy-2-naphthyl)propanoate. The group of renin-angiotensin system blocking agents (RAS blocking agents) includes ACE inhibitors, angiotensin receptor blockers (ARBs), selective and non selective beta blockers, and renin-inhibitors.

Renin-angiotensin system blocking agents can be taken alone or in combination with a diuretic.

Particularly suitable diuretics are selected from the group of sulfonamide derivatives preferably furosemide, or from the group of thiazide diuretics, for example chlorothiazide, chlorthalidone, hydrochlorothiazide, indapamide, metolazone, polythiazide.

The doses of 4-(nitrooxy)butyl(2S)-2-(6-methoxy-2-naphthyl)propanoate to be administered are determined depending upon, for example, age, body weight, symptoms, the desired therapeutic effects, the route of administration, and the treatment duration. In human adults, single doses administered range generally from 1 mg to 1000 mg, by oral administration, up to several times per day, and from 1 mg to 100 mg, by parenteral administration up to several times per day, or continuous administration for from 1 to 24 hours.

As mentioned above, the doses to be used depend upon various conditions. Therefore, there are cases wherein doses lower than or greater than the ranges specified above may be used.

The compound of the present invention may be administered in the form of, for example, solid compositions, liquid compositions or other compositions for oral administration, injections, liniments or suppositories for parenteral administration.

The present invention also provides pharmaceutical kits comprising 4-(nitrooxy)butyl(2S)-2-(6-methoxy-2-naphthyl)propanoate, a renin-angiotensin system blocking agent and optionally a diuretic, as combined preparation for simultaneous, separated or sequential use.

The synthesis of 4-(nitrooxy)butyl(2S)-2-(6-methoxy-2-naphthyl)propanoate is described in the WO 95/09831. The compound has the following formula:

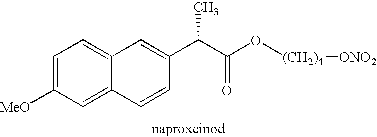

### EXPERIMENTAL PART

Effects of 4-(nitrooxy)butyl(2S)-2-(6-methoxy-2-naphthyl)propanoate (naproxcinod) and naproxen on systolic blood pressure (SBP).

The effects of two twice daily drug regimens (375 mg bid and 750 mg bid) of 4-(nitrooxy)butyl(2S)-2-(6-methoxy-2-naphthyl)propanoate (naproxcinod) on systolic blood pressure (SBP) vs. naproxen and placebo were studied in a 13-week double-blind, randomized clinical trial involving 207 osteoarthritic patients and hypertension treated with renin-angiotensin system blockers alone or in combination with a diuretic.

Patients were sequentially randomized in a 1:1:1:1 ratio using a validated remote automated system to receive for the 13-week study treatment period one of 4 treatment groups:
naproxcinod 750 mg twice daily,
naproxcinod 375 mg twice daily,
naproxen 500 mg (equimolar dose of naproxcinod 750 mg) twice daily, or
placebo twice daily.

The office SBP was measured in a standardized fashion by mercury column or aneroid device in supine position, at rest, at baseline and 2, 6, and 13 weeks after study treatment initiation. SBP measurements were to be performed 2 to 4 hours post-morning dose.

The comparability of the patients in the 4 treatment groups was determined from the demographic data and baseline BP values.

The data in Table I show that the difference in the mean change from baseline in systolic BP in the naproxcinod 750 mg bid group vs. naproxen 500 mg bid group was −6.5 mmHg.

These results demonstrate that naproxcinod induces less BP destabilization compared to naproxen and that it behaves similarly to placebo in patients with osteoarthritis and treated hypertension.

TABLE I
mean changes in systolic blood pressure (SBP)

| | | Difference in mean changes of SBP | | |
|---|---|---|---|---|
| | | (mmHg) mean ± SE | | |
| | Baseline | | | vs |
| | (mmHg) | vs | vs | naproxen |
| Groups | mean ± SD | baseline | placebo | 500 mg |
| placebo | 127.8 ± (12.8) | −4.6 ± 1.9 | — | 6.1 |
| naproxen | 127.8 ± (12.3) | 1.5 ± 1.9** | 6.1** | — |
| 500 mg | | | | |
| naproxcinod | 129.2 ± (15.0) | −2.7 ± 1.7 | 1.9 | −4.2 |
| 375 mg | | | | |
| naproxcinod | 129.0 ± (12.2) | −5.0 ± 1.7* | −0.4* | −6.5* |
| 750 mg | | | | |

*p < 0.02 vs naproxen 500 mg;
** p < 0.05 vs placebo

1. A method for the treatment of inflammation comprising administering to a patient with hypertension, who is co-administered with at least a renin-angiotensine system blocker, a therapeutically effective amount of 4-(nitrooxy)butyl(2S)-2-(6-methoxy-2-naphthyl)propanoate.

2. A method according to claim 1 wherein the inflammation is a musculo-skeletal disorder.

3. A method according to claim 2 wherein the musculo-skeletal disorders is osteoarthritis.

4. A method according to any of claim 1 wherein the patient is co-administered with a diuretic.
